(19) United States
(12) Patent Application Publication
(10) Pub. No.: US 2024/0016473 A1
(43) Pub. Date: Jan. 18, 2024

(54) IMPROVEMENTS IN OR RELATING TO THE GUIDING OF ELONGATE FLEXIBLE MEDICAL INSTRUMENTS

(71) Applicant: ROB'E GMBH, Hamburg (DE)

(72) Inventors: Jury SCHEWEL, Hamburg (DE); Dimitry SCHEWELL, Hamburg (DE)

(21) Appl. No.: 18/039,573

(22) PCT Filed: Dec. 1, 2021
(86) PCT No.: PCT/EP21/83720

§ 371 Date: May 31, 2023

(30) Foreign Application Priority Data
Dec. 1, 2020 (DE) 20 2020 005 003.2

(51) Int. Cl. A61B 8/12; A61B 8/08; A61B 8/00; A61M 25/01

(52) U.S. Cl. CPC A61B 8/12; A61B 8/0883; A61B 8/445; A61M 25/0136; A61B 8/4209

(57) ABSTRACT

In the field of echocardiography there is a need for improved guiding of elongate flexible medical instruments to shorten procedures and thereby reduce the exposure of an interventional cardiologist or cardiac surgeon, and an echocardiographer, to radioactive fluoroscopy.

A guide unit (10; 70; 150), for an elongate flexible medical instrument (12) steerable by an operator handle (60), comprises a guide base (14) that is fixedly securable in an operative position to define a reference datum (16) relative to an orifice into which the flexible medical instrument (12) is, in-use, to be inserted. The guide base (14) supports a guide assembly (28) which is engageable, in-use, with the flexible medical instrument (12) to selectively inhibit lengthwise axial movement of the flexible medical instrument (12) relative to the guide base (14). The guide assembly (28) is also moveable relative to the guide base (14) to allow rotation of the flexible medical instrument (12) about its axis (30) while inhibiting said lengthwise axial movement.

This invention relates to a guide unit for a flexible medical instrument which is steerable by an operator handle, a method of using such a guide unit, and a method of operating a flexible medical instrument inside a patient.

Echocardiography can be used in diagnostics, e.g. to provide images of intracardiac structures such as heart valves, left or right atria and ventricle, or the interatrial and interventricular septum.

Additionally, echocardiography can be used to assist in the performance of interventional or operative procedures, such as the treatment of structural heart disease or arrhythmias, by providing adequate visualization of the respective intracardiac structures to allow an interventional cardiologist or a surgeon, to accurately navigate various catheters, guide wires and other devices within a heart for optimal results.

Typically this requires an echocardiographer to both operate a flexible echocardiographic instrument, e.g. a transesophageal echocardiography (TEE) probe or an intracardiac echocardiography (ICE) catheter, within a patient orifice, while simultaneously operating an echocardiography display device to deliver optimal image quality. This requires a high degree of skill and ordinarily can be a time-consuming challenge which leads to longer procedures. Additionally procedures are becoming more complex which requires more time still to avoid reduced image quality which could otherwise lead to sub-optimal results.

Such lengthening of procedures is undesirable because it results in a dramatic increase. In exposure to radioactive fluoroscopy by both the surgeon, and particularly the echocardiographer.

According to a first aspect of the invention there is provided a guide unit, for an elongate flexible medical instrument steerable by an operator handle, comprising a guide base fixedly securable in an operative position to define a reference datum relative to an orifice into which the flexible medical instrument is in-use to be inserted, the guide base supporting a guide assembly engageable in-use with the flexible medical instrument to selectively inhibit lengthwise axial movement of the flexible medical instrument relative to the guide base, and the guide assembly being moveable relative to the guide base to allow rotation of the flexible medical instrument about its axis while inhibiting said lengthwise axial movement.

Having a guide unit which is able to define a reference datum relative to an orifice into which a steerable, flexible medical instrument, such as a flexible echocardiographic instrument, e.g. a transesophageal echocardiography (TEE) probe or an intracardiac echocardiography (ICE) catheter, is, in use, to be inserted is beneficial since it allows precise control of such insertion, e.g. Into a moving muscle such as an oesophagus, relative to the reference datum.

Moreover, additionally being able to selectively inhibit lengthwise axial movement of such a flexible medical instrument relative to the guide base, and hence relative in-use to the fixed reference datum, i.e. being able to selectively inhibit movement of the flexible medical instrument. In and out of the orifice, removes the need for a medical instrument operative to use one hand to perform such a task.

Furthermore, the inclusion of a guide assembly which is supported by, and moveable relative to, the guide base in a manner that allows rotation of the flexible medical instrument about its axis, while nevertheless continuing to inhibit the aforesaid lengthwise axial movement of the flexible medical instrument, is particularly beneficial. This is because it completely avoids the need for a medical instrument operative to use one hand to guide and fix in a lengthwise axial position the flexible medical instrument, and so de-skills to a considerable extent the process of inserting and operating the flexible medical instrument.

It follows that the guide unit of the invention, in use, improves the transmission of rotation of a flexible medical instrument about its own axis from an operator handle while also stabilising the position of the flexible medical instrument relative to a reference datum.

This allows such an operative to use only one hand to control the operator handle which steers the flexible medical instrument, e.g. Including rotating the flexible medical instrument about its own axis, and thereby frees up the other hand to perform another task, such as operating an echocardiography display device.

Still further, the provision of the guide unit of the invention provides the opportunity also to utilise automatic control of the said operator handle, e.g. by a second unit. In the form of a machine. This removes the need completely for a medical instrument operative to participate in the insertion and subsequent operation of the flexible medical instrument, and thereby permits such an operative to perform another task to speed up the overall time taken to complete a given flexible medical instrument procedure.

Preferably the guide assembly includes first and second opposed members to frictionally engage. In-use with the flexible medical instrument.

The first and second opposed members may be or include first and second rotatable members.

Such first and second members are readily able to provide the selective inhibition of lengthwise axial movement in a reliable and repeatable manner.

Optionally the first and second opposed members are moveable away from and towards one another.

Such movement advantageously facilitates loading, e.g. in use, of a flexible medical instrument between the first and second opposed members.

In a preferred embodiment of the invention the guide base and guide assembly are formed in first and second sub-assemblies which are moveable away from and towards one another.

Forming the guide base and guide assembly as two sub-assemblies, which are at least partially separable from one another, assists a medical instrument operative in readily loading a flexible medical instrument between the first and second opposed members, e.g. prior to use of the guide unit.

The first and second sub-assemblies may be pivotally coupled to one another.

Such an arrangement desirably allows movement of one sub-assembly away from and towards the other, while constraining such movement to ensure correct relative alignment of the sub-assemblies is maintained, e.g. when they are moved towards one another to, in use, engage the first and second opposed members with a flexible medical instrument.

Preferably the first and second sub-assemblies present a cover surface to one another. Having each sub-assembly include a cover surface, i.e. an internal cover that shields various components lying behind it, which it presents to the other sub-assembly desirably helps to prevent the ingress of biological contaminants, such as aerosol transmission from a flexible medical instrument guided thereby, into the respective sub-assemblies.

Optionally the first and second sub-assemblies are lockable. In abutment with one another.

Such a feature helps to ensure that the first and second sub-assemblies remain correctly aligned and orientated relative to one another, i.e. so that a flexible medical instrument guided thereby in use, remains desirably engaged with the guide assembly to ensure selective inhibition of any lengthwise axial movement of the flexible medical instrument.

In another preferred embodiment of the invention at least one of the first and second opposed members is driven by an actuator whereby in-use the flexible medical instrument is selectively moveable in a lengthwise axial direction relative to the guide base.

An ability to selectively move a flexible medical instrument in a lengthwise axial direction relative to the guide base, and hence relative in-use to the fixed reference datum, is advantageous because it allows the controlled movement of the flexible medical instrument inwards and outwards relative to the orifice into which it is inserted.

This, in turn, further reduces the need for input by one hand of an medical instrument operative to guide a flexible medical instrument, as well as selectively fix such an instrument. In lengthwise axial position or move such an instrument. In lengthwise axial direction.

Preferably one of the guide unit or the guide assembly is selectively moveable in a direction parallel to a lengthwise axial direction of a flexible medical instrument guided in-use thereby.

Such an arrangement allows for selective movement of a flexible medical instrument relative, in use, to the fixed reference datum, and thereby similarly permits the controlled movement of the flexible medical instrument inwards and outwards relative to the orifice into which. It is inserted.

The guide assembly may be or include a turntable which is rotatable relative to the guide base.

The provision of such turntable advantageously permits the necessary movement, e.g. rotation, of the guide assembly relative to the guide base to allow, in use, a flexible medical instrument that is being guided by the guide unit to rotate about its axis, and therefore continue to be steerable by an associated operator handle coupled therewith.

Optionally one or more of the guide base and the guide assembly, as well as any sub-assemblies or parts thereof, are selectively completely separable from one another.

Such an arrangement permits the ready replacement of one or more such assemblies, sub-assemblies or parts with an identical replacement assembly, sub-assembly or part in order to avoid the need to clean and sterilise the original, replacement assembly, sub-assembly or part, and thereby reduce the dwell time between one use of the guide unit and another.

A guide unit according to a still further preferred embodiment of the invention further includes a mounting member to fixedly secure the guide base in its operative position.

Including such a mounting member desirably facilitates the ready fixing of the guide base to an essentially stationary object, such as a patient, an operating table, the wall or roof of an operating room or an item of medical equipment likely to be found in an operating room, e.g. an ultrasound machine, an X-ray system, a fluoroscopy device such as a C-arm, an oxygen or ventilator machine, or an infusion or transfusion stand. In this way the guide base can be easily fixed in an operative position. In which. It defines a suitable reference datum.

Preferably the mounting member houses one or more of at least one power cable to supply power to the guide unit, and at least one control cable to permit control of the guide unit.

Housing one or more such cables, i.e. within, the mounting member advantageously protects such elements from biological contaminants and thereby helps to make the invention easier to clean and sterilise between uses.

According to a second aspect of the invention there is provided a method of using a guide unit according to any preceding claim comprising the steps of:
(a) fixedly securing the guide base in an operative position to define a reference datum relative to an orifice into which a flexible medical instrument is to be inserted;
(b) engaging the guide assembly with the flexible medical instrument; and
(c) steering the flexible medical instrument using an operator handle.

According to a third aspect of the invention there is provided a method of operating a flexible medical instrument inside a patient comprising the steps of:
(a) providing a guide unit according to any of Claims 1 to 14;
(b) fixedly securing the guide base. In an operative position to define a reference datum relative to a patient orifice;
(c) Inserting the flexible medical instrument into the patient orifice;
(d) engaging the guide assembly with the flexible medical instrument; and
(e) steering the flexible medical instrument using an operator handle.

The methods of the invention share the benefits of the corresponding features of the guide unit of the invention.

It will be appreciated that the use of the terms “first” and “second”, and the like, in this patent specification is merely intended to help distinguish between similar features (e.g. the first and second transmission conduits, and the first and second power converters), and is not intended to indicate the relative importance of one feature over another feature, unless otherwise specified.

Within the scope of this application it is expressly intended that the various aspects, embodiments, examples and alternatives set out. In the preceding paragraphs, and the claims and/or the following description and drawings, and in particular the individual features thereof, may be taken independently or in any combination. That is, all embodiments and all features of any embodiment can be combined in any way and/or combination, unless such features are incompatible. The applicant reserves the right to change any originally filed claim or file any new claim accordingly, including the right to amend any originally filed claim to depend from and/or incorporate any feature of any other claim although not originally claimed in that manner.

There now follows a brief description of preferred embodiments of the invention, by way of non-limiting example, with reference being made to the following figures in which:

FIG. 1 shows a guide unit according to a first embodiment of the invention;

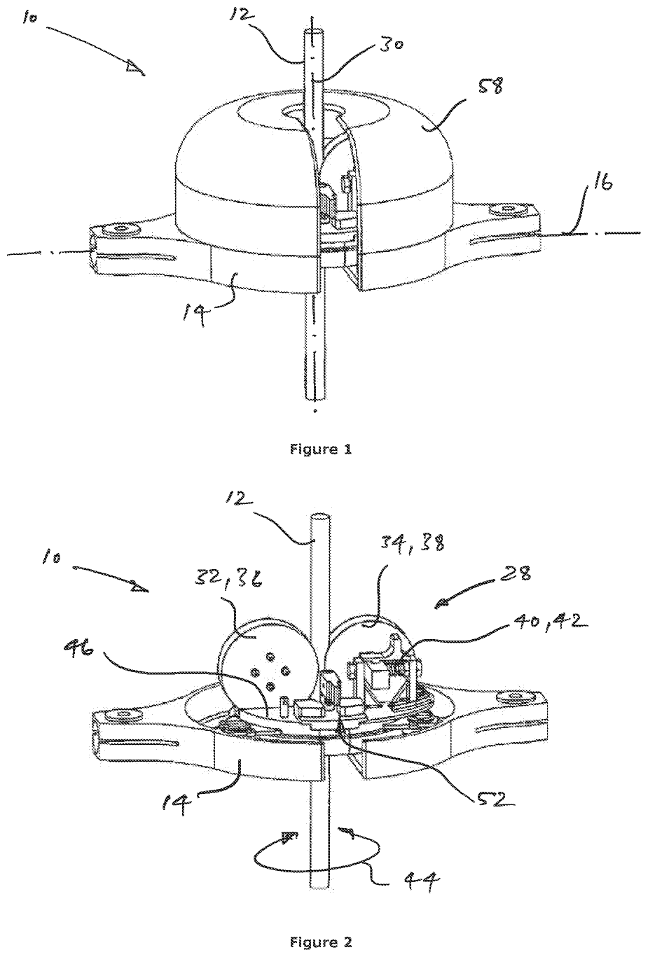

FIG. 2 shows interior features of the first guide unit shown in FIG. 1;

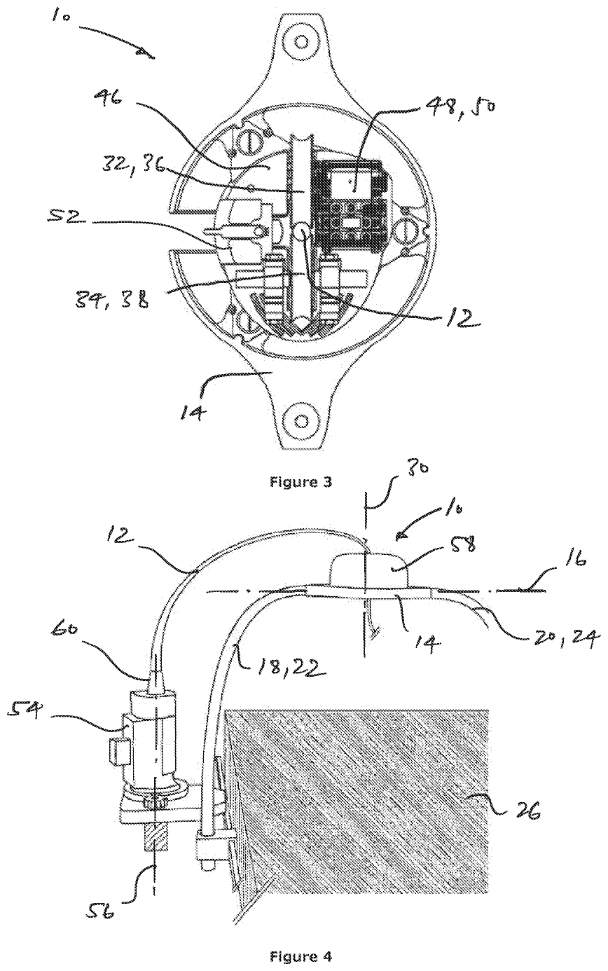

FIG. 3 shows a plan view from of the interior feature of the first guide unit;

FIG. 4 shows the first guide unit fixedly secured in an operative position;

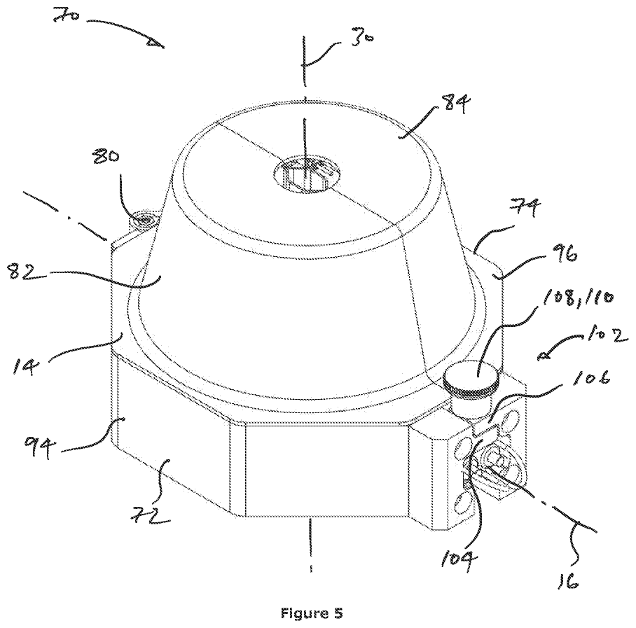

FIG. 5 shows a guide unit according to a second embodiment of the invention in a first, closed configuration;

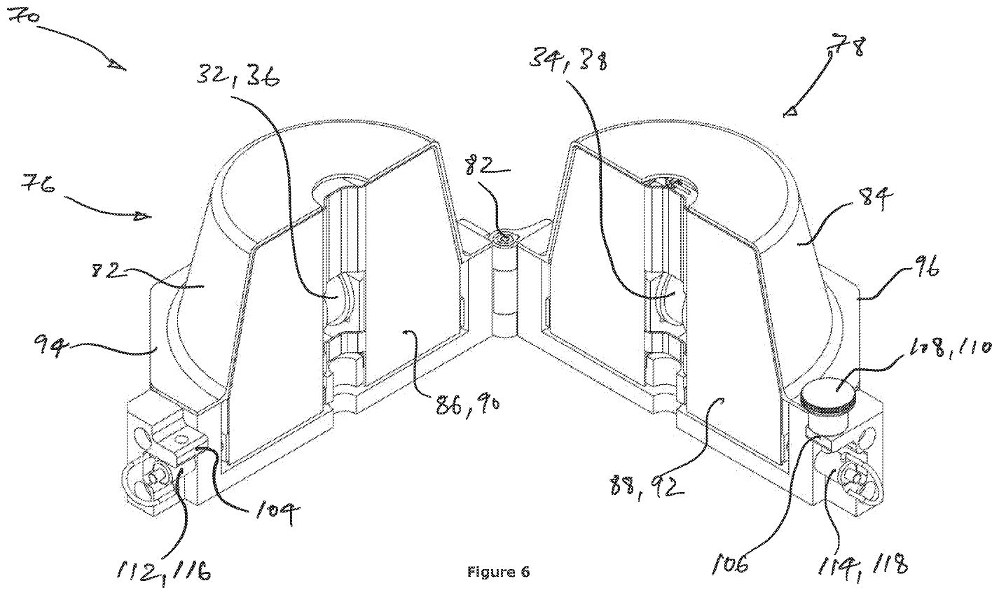

FIG. 6 shows the second guide unit shown in FIG. 5 in a second, open configuration;

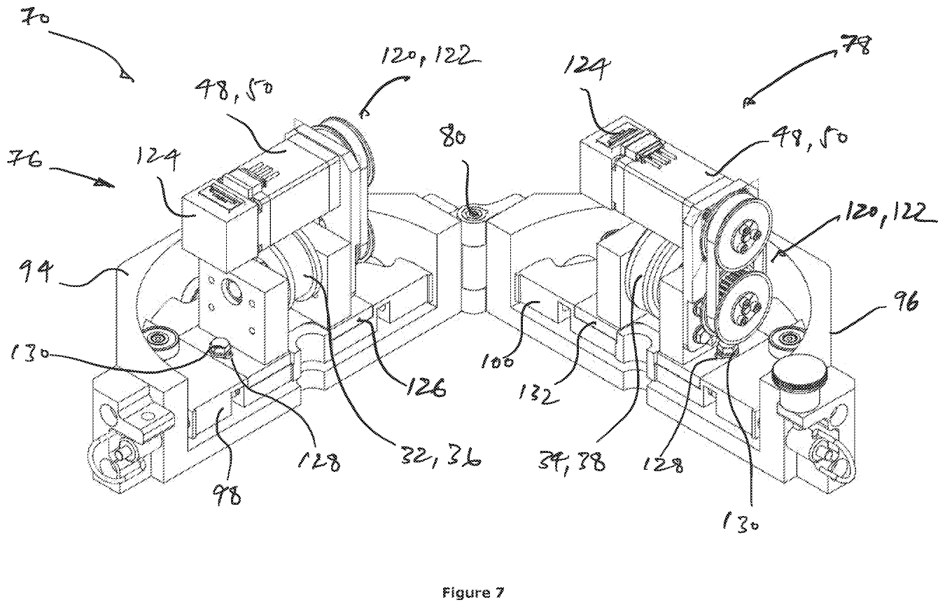

FIG. 7 shows interior features of the second guide unit;

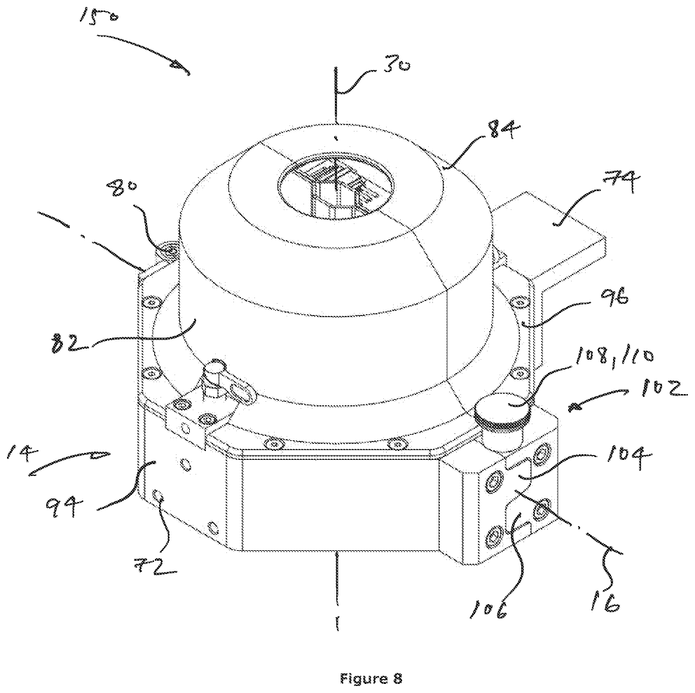

FIG. 8 shows a guide unit according to a third embodiment of the invention. In a first, closed configuration;

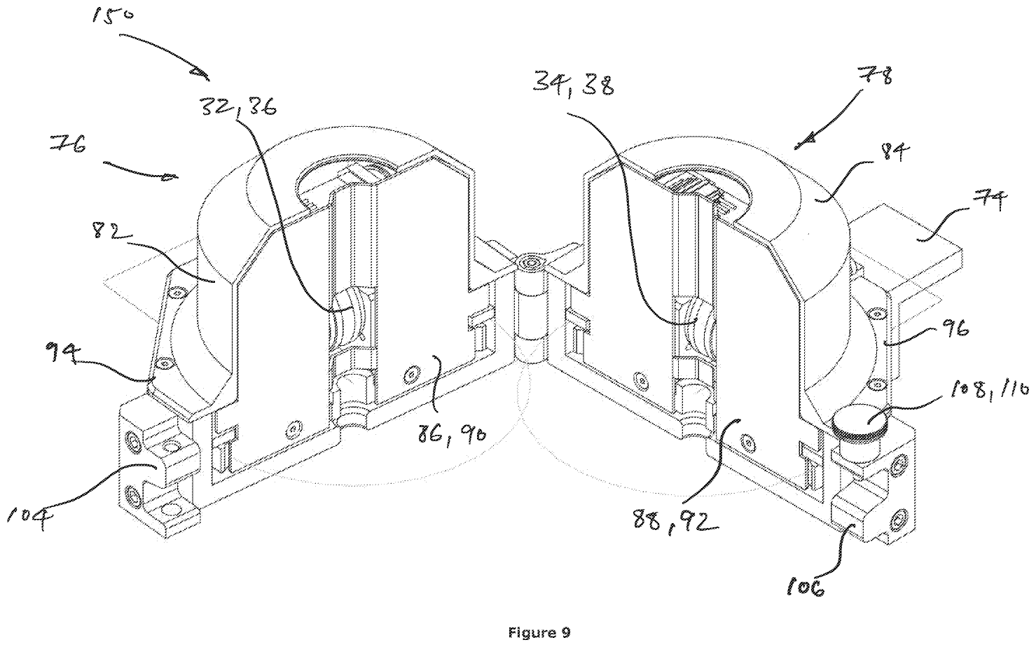

FIG. 9 shows the third guide unit shown. In FIG. 8 in a second, open configuration;

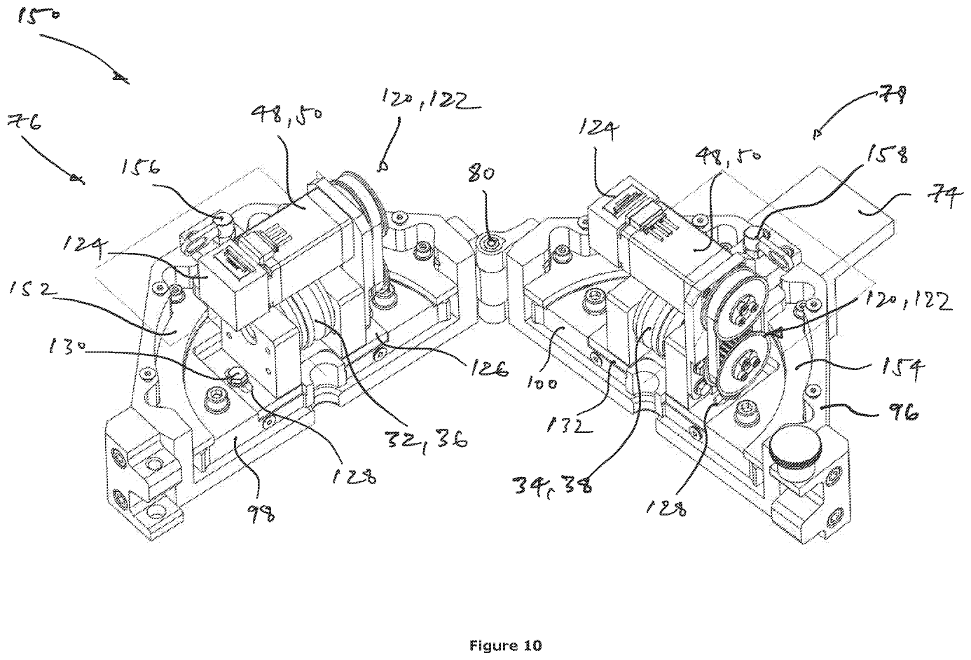

FIG. 10 shows interior features of the third guide unit.

A guide unit according to a first embodiment of the invention is designated generally by reference numeral 10, as shown. In FIGS. 1 to 4.

The first guide unit 10 is for guiding an elongate flexible medical instrument 12 (only a part of which is shown. In the figures) which is steerable by an operator handle 60 (only a part of which is shown in FIG. 4). Such a flexible medical instrument 12 could be a flexible echocardiographic instrument, e.g. a transesophageal echocardiography (TEE) probe or an intracardiac echocardiography (ICE) catheter, as well as any other types of sonographic or echocardiographic probe, device, catheter or instrument, which can be used for medical or non-medical purposes.

The first guide unit 10 is a discrete, separate unit which allows. It to be easily positioned where needed. In particular, the first guide unit 10 includes a guide base 14 which, in use, is fixedly securable, i.e. can be fixed in a manner whereby it does not readily or easily move, in an operative position in order to define a reference datum 16, such as that shown in FIG. 3. The reference datum 16 is with respect to an orifice (not shown), e.g. a patient's mouth and associated oesophagus, into which the flexible medical instrument 12 is, in use, to be inserted, and so the guide base 14 may be positioned over the head of a patient (not shown), e.g. In front of or within a patient's mouth.

In the embodiment shown, the first guide unit 10 includes first and second mounting members 18, 20 in the form of first and second arms 22, 24, each of which is coupled with the guide base 14 and which together define a mounting mechanism. Each arm 22, 24 is mounted on, e.g. fixedly secured to, an operating table 26 to hold the guide base in the aforesaid operative position. However, one or more of the arms may instead be fixed to another form of essentially stationary object, such as a patient, the wall or roof of an operating room or an item of medical equipment likely to be found in an operating room, e.g. an ultrasound machine, an X-ray system, a fluoroscopy device such as a C-arm, an oxygen or ventilator machine, or an infusion or transfusion stand.

Other embodiments of the invention may include only one mounting member, e.g. only one arm, fixed in one of the manners set out above. In any event, however, preferably at least one such mounting member houses at least one power cable to supply power to the guide unit, and/or at least one control cable to permit control of the guide unit.

Returning to the embodiment shown, the guide base 14 supports a guide assembly 28 which is engageable, in-use, with a flexible medical instrument 12 to selectively inhibit lengthwise axial movement of the flexible medical instrument 12 relative to the guide base 14, i.e. selectively inhibit movement in a direction along the elongate axis 30 of the flexible medical instrument 12.

More particularly, the guide assembly 28 includes first and second opposed members 32, 34 which, in use, frictionally engage with the flexible medical instrument 12. More particularly still, each of the first and second opposed members 32, 34 is a respective rotatable member 36, 38 in the form a wheel which is mounted within the guide assembly 28. Each rotatable member 36, 38 has an outer peripheral profile that is complementary to the cross-sectional shape of the flexible medical instrument 12, e.g. circular in the embodiment shown. Other forms of first and second opposed member, as well as other types of rotatable member may also be used, however. Moreover, any first or second opposed member may be formed from a resiliently deformable material, such as a rubber material, which usefully helps to accommodate differing diameters of flexible medical instrument.

The first and second opposed members 32, 34, i.e. the first and second rotatable members 36, 38, are moveable away from and towards one another. In the embodiment shown, the second rotatable member 38 is moveably mounted, e.g. slidably mounted, within the guide assembly 28 and resiliently biased, e.g. using a biasing member 40 such as a spring 42, towards the first rotatable member 36. In this manner, the second rotatable member 38 is moveable away from the first rotatable member 36, e.g. to allow insertion of a flexible medical instrument 12 therebetween, after which the biasing member 40, i.e. spring 42, urges the second rotatable member 38 towards the first rotatable member 36 and thereby frictionally engages the flexible medical instrument 12 between the first and second rotatable members 36, 38, e.g. by applying a clamping force to the flexible medical instrument 12.

In addition to the foregoing, the guide assembly 28 is also moveable relative to the guide base 14 to allow rotation 44 in a circumferential direction of the flexible medical instrument 12 about its axis, i.e. Its elongate axis 30.

More particularly, the guide assembly 28 includes a turntable 46 which is rotatable relative to the guide base 14.

The turntable 46, while being able to rotate relative to the guide base 14, is mounted within the guide base 14 in a manner that inhibits movement of the turntable 46 in a lengthwise axial direction, i.e. In a direction along the elongate axis 30 of the flexible medical instrument 12. As a consequence the turntable 46, and hence the guide assembly 28 of which the turntable 46 is a part, allows rotational movement 44 of the flexible medical instrument 12 about its elongate axis 30, while at the same time inhibiting lengthwise axial movement, i.e. movement in a direction along the elongate axis 30, of the flexible medical instrument 12 relative to the guide base 14, and thus relative to the reference datum 16.

In use, this permits the first guide unit 10 to allow rotation 44 of the flexible medical instrument 12 about its elongate axis 30, so as to permit operation and movement of the flexible medical instrument 12, e.g. within a patient, while selectively preventing movement of the flexible medical instrument 12 in to or out of, e.g. an orifice of such a patient.

Additionally, however, the first opposed member 32, i.e. the first rotatable member 36, is driven by an actuator 48 which, in the embodiment shown, is an electric motor 50 (as best shown in FIG. 3), although other types of actuator, i.e. any device which converts energy into motion, may be used.

The further inclusion of such an actuator 48, i.e. motor 50, allows the first rotatable member 36 to selectively move the flexible medical instrument 12 in a lengthwise axial direction relative to the guide base 14, and thus, in use, selectively move the flexible medical instrument 12 in to or out of an orifice, e.g. of a patient.

The actuator 48, i.e. motor 50, may provide position data, e.g. regarding the speed and extent of rotation, such that a control unit is able to determine the extent of lengthwise axial movement of the flexible medical instrument 12 relative to the guide base 14, i.e. is able to determine the extent to which the flexible medical instrument 12 moves into or out of a corresponding orifice.

In other embodiments of the invention, each of the first and second opposed members 32, 34, i.e. each of the first and second rotatable members 36, 38 may be driven by an actuator, e.g. a motor, so as to increase the driving force which can be applied to the flexible medical instrument 12.

Also, in still further other embodiments of the invention one of the first guide unit or the guide assembly is selectively moveable in a direction parallel to the elongate axis of the flexible medical instrument so as to achieve similar lengthwise axial movement of the flexible medical instrument relative to the reference datum, i.e. so as to similarly effect, in use, movement of the flexible medical instrument into and out of an orifice.

Meanwhile, returning to the embodiment shown, the first guide assembly 28 also includes a retention assembly 52 which is moveable between a first, closed position (as shown in the figures) In which access to a region between the first and second opposed members 32, 34, i.e. the first and second rotatable members 36, 38, is inhibited, and a second, open position (not shown) in which access to the said region is possible.

Movement of the retention assembly 52 into the second, open position thereby allows access, e.g. by a medical instrument operative, to the region between the first and second rotatable members 36, 38, and thus permits the operative to move the second rotatable member 38 away from the first rotatable member 36, i.e. against the bias provided by the spring 42, to, in use, load a flexible medical instrument 12 between the first and second rotatable members 36, 38, and thereby have the first and second rotatable member 36, 38 frictionally engage with the flexible medical instrument.

Additionally, the first guide unit 10 may include a guide housing 58, as best shown in FIG. 1 (and as is shown removed in FIGS. 2 and 3), to both protect the guide assembly 28 from damage and present a more easily cleaned exterior.

Moreover, one or more of the guide base 14 and the guide assembly 28, as well as any sub-assemblies or parts thereof, such as each rotatable member 36, 38 (and combination with the actuator 48, i.e. motor 50, in the case of the first rotatable member), may be completely separable from one another, if desired, e.g. so as to facilitate replacement with an identical component so as to avoid the need to clean and sterilise the replaced component.

In use of the first guide unit 10 a user, e.g. a medical instrument operative such as an echocardiographer, first fixedly secures the guide base 14 in an operative position, e.g. by fixing each arm 22, 24 to an operating table 26, so as to define a reference datum 16 relative to an orifice into which a flexible medical instrument 12 is to be inserted.

The user then moves the retention assembly 52 into its second, open position to allow access to the first and second rotatable members 36, 38, and more particularly to allow the user to move the second rotatable member 38 away from the first rotatable member 36, against the bias of the spring 42, and thereafter to engage the guide assembly 28 with a flexible medical instrument 12 by inserting the flexible medical instrument 12 between the first and second rotatable members 36, 38.

The user is able then to steer the flexible medical instrument 12 using an operator handle 60.

One way. In which the user may achieve such steering of the flexible medical instrument 12 is by way of a base unit 54, i.e. a second unit (with the guide unit 10 thereby being a first unit), which is configured to selectively rotate the entire operator handle 60 around its own handle axis 56, as shown in FIG. 4. Such rotational movement of the operator handle 60 translates into rotational movement 44 of the flexible medical instrument 12 (and the guide assembly 28 it is engaged with) relative to the guide base 14, and thereby, in use, rotation of the flexible medical instrument 12, about its own axis 30 within, e.g. a patient.

The base unit 54 may additionally include first and second base unit motors (not shown) to selectively rotate each of a first, small operator wheel and a second, large operator wheel (neither shown) on the operator handle 60 to manipulate a distal end (not shown) of the flexible medical instrument 12, in a manner known. In the art.

A guide unit according to a second embodiment of the invention is designated generally by reference numeral 70, as shown in FIGS. 5 to 7.

The second guide unit 70 is again for guiding an elongate flexible medical instrument (not shown) which is steerable by an operator handle (not shown), and as such the second guide unit 70 shares many features with the first guide unit 10. Such like features share the same reference numerals.

Accordingly, the second guide unit 70 includes a guide base 14 which, in use, is also fixedly securable in an operative position in order to define a reference datum.

The second guide unit 70 includes first and second engagement formations 72, 74, one or more of which is securable to a mounting member (not shown) to permit such securing of the second guide unit 70 in an operative position. The or each such mounting member may similarly house one or more power cables and/or control cables.

The guide base 14 again supports a guide assembly 28 which is engageable, in-use, with a flexible medical instrument to selectively inhibit lengthwise axial movement of the flexible medical instrument relative to the guide base 14, i.e. again selectively inhibit movement in a direction along the elongate axis of the flexible medical instrument.

The guide assembly 28 of the second guide unit 70 again includes first and second opposed members 32, 34 which, in use, frictionally engage with the flexible medical instrument, and each of the first and second opposed members 32, 34 is again a respective rotatable member 36, 38 in the form a wheel which is mounted within the guide assembly 28. Each rotatable member 36, 38 similarly has an outer peripheral profile that is complementary to the cross-sectional shape of the flexible medical instrument, e.g. circular in the embodiment shown. Other forms of first and second opposed member, as well as other types of rotatable member may also be used. In relation to the second guide unit, however. Moreover, any such first and second opposed member, e.g. respective rotatable member, may be formed from a resiliently deformable material to help accommodate flexible medical instruments having different diameters.

The first and second opposed members 32, 34, i.e. the first and second rotatable members 36, 38, are again moveable away from and towards one another to permit loading of a flexible medical instrument therebetween.

However, one way in which the second guide unit 70 differs from the first guide unit 10 is that the guide base 14 and the guide assembly 28 are formed as first and second sub-assemblies 76, 78 which are moveable away from and towards one another. More particularly, the first and second sub-assemblies are pivotally connected to one another, e.g. via a hinge 80, although other forms of pivotal connection are also possible.

Such separation of the guide base 14 and the guide assembly 28 into first and second sub-assemblies 76, 78 necessarily requires the guide housing of the second guide unit 70 to be formed from first and second guide housing parts 82, 84.

In a similar manner, each sub-assembly 76, 78 includes a corresponding first or second inner housing face 86, 88, each of which defines a cover surface 90, 92. The inclusion of such first and second inner housing faces 86, 88 permits the first and second sub-assemblies 76, 78 to present a corresponding cover surface 90, 92 to one another, e.g. when moving from a second, open configuration (as shown in FIG. 6) in which loading of a flexible medical instrument between the first and second opposed members 32, 34, i.e. between the first and second rotatable members 36, 38, is possible, towards a first, closed configuration (as shown in FIG. 5) in which the first and second rotatable members 36, 38 engage, in use, with said flexible medical instrument and thereby permit selective inhibition of any lengthwise axial movement of the flexible medical instrument relative to the guide base 14. The cover surfaces 90, 92 shown are substantially planar, although this need not be the case. Preferably, however, the cover surfaces are complementary in shape to one another.

In a similar manner, the guide base of the second guide unit 70 differs from that of the first guide unit 10 in that it is formed from first and second guide base parts 94, 96, each of which forms a part of the corresponding first or second sub-assembly 76, 78 so as to facilitate the aforesaid pivotal movement of one sub-assembly 76, 78 away from and towards the other sub-assembly 76, 78.

Meanwhile, the guide assembly 28 of the second guide unit 70 again includes a turntable 46 which is rotatable relative to the guide base 14 in order to allow rotation of the guide assembly 28 relative to the guide base 14, and thereby again permit rotation 44 in a circumferential direction of a flexible medical instrument about its axis 30 while otherwise being retained and restrained by the guide assembly 28.

The turntable 46 of the second guide unit 70 likewise differs from that of the first guide unit 10 in that it is formed from first and second turntable parts 98, 100, each of which forms a part of the corresponding first or second sub-assembly 76, 78.

The turntable parts 94, 96, while being able to rotate relative to the guide base 14, are mounted within the guide base 14 in a manner that inhibits movement of each turntable part 98, 100 in a lengthwise axial direction, i.e. In a direction along the elongate axis 30 of a flexible medical instrument retained thereby.

As a consequence the turntable parts 98, 100, and hence the guide assembly 28 of which the turntable parts 98, 100 are a part, allows rotational movement 44 of such a flexible medical instrument about its elongate axis 30, while at the same time inhibiting lengthwise axial movement, i.e. movement in a direction along the elongate axis 30, of the flexible medical instrument relative to the guide base 14, i.e. the respective guide base parts 94, 96, and thus relative to a desired reference datum.

In use, this similarly permits the second guide unit 70 to allow rotation 44 of a flexible medical instrument about its elongate axis, so as to permit operation and movement of the flexible medical instrument, e.g. within a patient, while selectively preventing movement of the flexible medical instrument in to or out of, e.g. an orifice of such a patient.

The second guide unit 70 additionally includes a locking assembly 102 to lock the first and second sub-assemblies 76, 78 in abutment with one another, i.e. in first, closed position as shown in FIG. 5, to permit such use of the second guide unit 70.

In the preferred embodiment shown, the locking assembly 102 includes mutually co-operable locking formations 104, 106 on the respective first and second guide assembly parts 94, 96, which are held together by a locking member 108 in the form of a pin 110, although other types and configurations of locking assembly may also be used.

Additionally, each guide base part 94, 96 includes a corresponding retention member 112, 114, optionally in the form of a respective retention pin 116, 118, to selectively lock each corresponding turntable part 98, 100 in position, e.g. as shown in FIG. 7. Such temporary locking of the turntable parts 98, 100 in position is desirable, for example, when wishing to move the first and second sub-assemblies 76, 78 into the second, open position, e.g. as shown in FIG. 6.

A further difference in the second guide unit 70 is that each of the first and second opposed members 32, 34, i.e. each of the first and second rotatable members 36, 38 is driven by an actuator 48, although this need not necessarily be the case. Each actuator is again optionally an electric motor 50, although other types of actuator are possible.

More particularly each rotatable member 36, 38 is coupled with a corresponding actuator 48, i.e. a corresponding motor 50, via drive assembly 120 in the form of a pulley and gear wheel combination 122, although other types of drive assembly are possible, and indeed may be disposed of in some embodiments.

The inclusion of such actuators 48, i.e. motors 50, similarly allows each of the first and second rotatable members 36, 38 to selectively move a flexible medical instrument. In a lengthwise axial direction relative to the guide base 14, and thus, in use, selectively move the flexible medical instrument in to or out of an orifice, e.g. of a patient.

Each actuator 48, i.e. each motor 50, includes an encoder 124 to provide position data, e.g. regarding the speed and extent of rotation of each rotatable member 36, 38.

In addition to the foregoing, each of the first and second opposed members 32, 34, i.e. each of the first and second rotatable members 36, 38, is moveably mounted relative to the corresponding turntable part 98, 100, and more particularly are moveably mounted towards and away from one another while the first and second sub-assemblies 76, 78 are in their first, closed position.

In that regard, the first opposed member 32, i.e. the first rotatable member 36, is mounted on a first platform 126 which is slidable relative to the first turntable part 98. The extent of relative slidable movement is constrained by first and second slots 128 (only one of which is shown) formed in the first platform 126, each of which cooperates with a corresponding first and second abutment 130 (only one of which is shown) that is secured to the first turntable part 98. Similarly, the second opposed member 34, i.e. the second rotatable member 38, is mounted on a second platform 132 which is slidable relative to the second turntable part 100, with the extent of relative slidable movement again being constrained by first and second slots 128 which cooperate with corresponding first and second abutments 130.

Additionally, the first and second platforms 126, 132 are biased towards one another, e.g. by a spring (not shown).

Including such moveably mounted and biased first and second opposed member 32, 34 desirably allows them to accommodate therebetween flexible medical instruments 12 of differing diameters.

Also, in other embodiments of the invention one of the second guide unit or its guide assembly may be selectively moveable in a direction parallel to the elongate axis of a flexible medical instrument retained thereby so as to achieve similar lengthwise axial movement of the flexible medical instrument relative to the reference datum, i.e. so as to similarly effect, in use, movement of the flexible medical instrument into and out of an orifice.

One or more of the guide base 14, i.e. each guide base part 94, 96, and the guide assembly 28, i.e. each turntable part 98, 100, associated rotatable member 36, 38 and corresponding motor 50, may be completely separable from one another, if desired, e.g. so as to facilitate replacement with an identical component so as to avoid the need to clean and sterilise the replaced component.

In use of the second guide unit 70 a user, e.g. a medical instrument operative such as an echocardiographer, again first fixedly secures the guide base 14 in an operative position, so as to define a reference datum relative to an orifice into which a flexible medical instrument (not shown) is to be inserted.

The user then moves the first and second sub-assemblies 76, 78 into their second, open position (i.e. as shown in FIGS. 6, and 7), to allow insertion of a flexible medical instrument between the first and second rotatable members 36, 38, before returning the first and second sub-assemblies 76, 78 to their first, closed position (i.e. as shown. In FIG. 5) in which the first and second rotatable members 36, 38, and hence the guide assembly 28 of the second guide unit 70, are engaged with a flexible medical instrument.

The user is able then to steer the flexible medical instrument using an operator handle (not shown), either manually using one hand, or automatically, e.g. using a second base unit 54, such as the one described. In combination with the first guide unit 10. A guide unit according to a third embodiment of the invention is designated generally by reference numeral 150, as shown in FIGS. 8 to 10.

The third guide unit 150 is again for guiding an elongate flexible medical instrument (not shown) which is steerable by an operator handle (not shown), and is very similar to second guide unit 70, with such like features sharing the same reference numerals.

Accordingly, the third guide unit 150 includes first and second engagement formations 72, 74, only the second of which is shown completely. One or more of such engagement formations 72, 74 is securable to a mounting member (not shown) to permit securing of the third guide unit 150 in an operative position.

The third guide unit 150 similarly includes a guide base 14 and a guide assembly 28 that are formed as first and second sub-assemblies 76, 78 which are moveable away from and towards one another and, more particularly, are again pivotally connected to one another, e.g. via a hinge 80, although other forms of pivotal connection are also possible.

The turntable 46 of the third guide unit 150 likewise is formed from first and second turntable parts 98, 100, each of which forms a part of a corresponding first or second sub-assembly 76, 78.

The turntable parts 94, 96, while being able to rotate relative to the guide base 14, are mounted within the guide base 14 in a manner that inhibits movement of each turntable part 98, 100 in a lengthwise axial direction, i.e. in a direction along the elongate axis 30 of a flexible medical instrument retained thereby. Such movement in a lengthwise axial direction is inhibited in the third guide unit 150 by the inclusion of first and second retaining plates 152, 154, each of which is mounted over a corresponding first or second turntable part 98, 100 to allow rotational movement of the respective turntable part 98, 100 relative to the corresponding guide base part 94, 96 but prevent relative movement in the lengthwise axial direction.

As a consequence the turntable parts 98, 100, and hence the guide assembly 28 of which the turntable parts 98, 100 are a part, allows rotational movement 44 of such a flexible medical instrument about its elongate axis 30, while at the same time inhibiting lengthwise axial movement, i.e. movement. In a direction along the elongate axis 30, of the flexible medical instrument relative to the guide base 14, i.e. the respective guide base parts 94, 96, and thus relative to a desired reference datum.

In use, this similarly permits the third guide unit 150 to allow rotation 44 of a flexible medical instrument about its elongate axis, so as to permit operation and movement of the flexible medical instrument, e.g. within a patient, while selectively preventing movement of the flexible medical instrument in to or out of, e.g. an orifice of such a patient.

One way in which the third guide unit 150 differs from the second guide unit 70 is that the retention member 112, 114 included in each guide base part 94, 96 takes the form of a respective retention clamp 156, 158, rather than a respective retention pin 116, 118.

Each respective retention clamp 156, 158 again selectively locks each corresponding turntable part 98, 100 in position, e.g. as shown in FIG. 10, but does so by way of being driven by a complementation screw thread formation into engagement with the corresponding turntable part 98, 100. In addition, preferably each turntable part 98, 100 includes at least one receiving formation (not shown) to receive a portion of the corresponding retention clamp 156, 158. In this manner, cooperation of the receiving formation and respective retention clamp 156, 158 can be used to selectively lock a given turntable part 98, 100 in a predetermined position, e.g. as shown in FIG. 10, relative to the corresponding guide base part 94, 96. Suitable positioning of a turntable part 98, 100 relative to the corresponding guide base part 94, 96 may be confirmed by a visual indicator, e.g. the alignment of two reference marks (not shown).

The third guide unit 150 may be used in essentially the same manner as the second guide unit 70.

1. A guide unit, for an elongate flexible medical instrument steerable by an operator handle, comprising a guide base fixedly securable in an operative position to define a reference datum relative to an orifice into which the flexible medical instrument is in-use to be inserted, the guide base supporting a guide assembly engageable in-use with the flexible medical instrument to selectively inhibit lengthwise axial movement of the flexible medical instrument relative to the guide base, and the guide assembly being moveable relative to the guide base to allow rotation of the flexible medical instrument about its axis while inhibiting said lengthwise axial movement.

2. A guide unit according to claim 1 wherein the guide assembly includes first and second opposed members to frictionally engage in-use with the flexible medical instrument.

3. A guide unit according to claim 2 wherein the first and second opposed members are or include first and second rotatable members.

4. A guide unit according to claim 2 wherein the first and second opposed members are moveable away from and towards one another.

5. A guide unit according to claim 4 wherein the guide base and guide assembly are formed in first and second sub-assemblies which are moveable away from and towards one another.

6. A guide unit according to claim 5 wherein the first and second sub-assemblies are pivotally coupled to one another.

7. A guide unit according to claim 5 wherein the first and second sub-assemblies present a cover surface to one another.

8. A guide unit according to claim 5 wherein the first and second sub-assemblies are lockable in abutment with one another.

9. A guide unit according to claim 2 wherein at least one of the first and second opposed members is driven by an actuator whereby in-use the flexible medical instrument is selectively moveable in a lengthwise axial direction relative to the guide base.

10. A guide unit according to any of claim 2 wherein one of the guide unit or the guide assembly is selectively moveable in a direction parallel to a lengthwise axial direction of a flexible medical instrument guided in-use thereby.

11. A guide unit according to any preceding claim wherein the guide assembly is or includes a turntable which is rotatable relative to the guide base.

12. A guide unit according to claim 1 wherein one or more of the guide base and the guide assembly, as well as any sub-assemblies or parts thereof, are selectively completely separable from one another.

13. A guide unit according to claim 1 further including a mounting member to fixedly secure the guide base in its operative position.

14. A guide unit according to claim 13 wherein the mounting member houses one or more of at least one power cable to supply power to the guide unit, and at least one control cable to permit control of the guide unit.

15. A method of using a guide unit according to claim 1 comprising the steps of:
(a) fixedly securing the guide base in an operative position to define a reference datum relative to an orifice into which a flexible medical instrument is to be inserted;
(b) engaging the guide assembly with the flexible medical instrument; and
(c) steering the flexible medical instrument using an operator handle.

16. A method of operating a flexible medical instrument inside a patient comprising the steps of:
(a) providing a guide unit for an elongate flexible medical instrument steerable by an operator handle, comprising a guide base fixedly securable in an operative position to define a reference datum relative to an orifice into which the flexible medical instrument is in-use to be inserted, the guide base supporting a guide assembly engageable in-use with the flexible medical instrument to selectively inhibit lengthwise axial movement of the flexible medical instrument relative to the guide base, and the guide assembly being moveable relative to the guide base to allow rotation of the flexible medical instrument about its axis while inhibiting said lengthwise axial movement;
(b) fixedly securing the guide base in an operative position to define a reference datum relative to a patient orifice;
(c) inserting the flexible medical instrument into the patient orifice;
(d) engaging the guide assembly with the flexible medical instrument; and
(e) steering the flexible medical instrument using an operator handle.
